(19) United States
(12) Patent Application Publication
(10) Pub. No.: US 2008/0124720 A1
(43) Pub. Date: May 29, 2008

(54) Method Of Sample Analysis And Apparatus Therefor

(75) Inventors: Stephen J. Sowerby, Dunedin (NZ); Murray Frederick Broom, Dunedin (NZ); Richard Brian McCaslin, Wellesley, MA (US)

Correspondence Address: FOLEY & LARDNER LLP, 111 HUNTINGTON AVENUE, 26TH FLOOR, BOSTON, MA, 02199-7610, US

(73) Assignee: GLOBAL TECHNOLOGIES(NZ) LTD., DUNEDIN (NZ)

(21) Appl. No.: 11/720,387

(22) PCT Filed: Nov. 30, 2004
(86) PCT No.: PCT/NZ2004/000311

§ 371 Date: Sep. 11, 2007

(51) Int. Cl. C12Q 1/68; G01N 33/00; C12Q 1/00; B01J 19/00; G01N 21/75

(52) U.S. Cl. 435/6; 436/183; 436/94; 436/63; 435/4; 436/166; 422/68.1

(57) ABSTRACT

A method and apparatus for processing and analysing samples. The invention can be used in relation to any sample analysis where processing of the sample with one or more reagents is required. The invention has particular relevance for the DNA analysis of biological materials. The method includes introducing the sample into a conduit (10), where the conduit (10) has at least one encapsulated aqueous solution of one or more sample processing reagents (13) and where each encapsulated aqueous solution (13) is encapsulated by a solid or semi-solid hydrophobic material (12); applying heat so that the solid or semi-solid material (12) liquefies allowing the sample and/or the at least one encapsulated aqueous solution (13) to move along the conduit (10); causing the aqueous solution of one or more sample processing reagents (13) to come into contact with the sample and react with the sample to form a product mixture; and detecting the presence, and optionally measuring the concentration, of a product in the product mixture.

### FIELD OF INVENTION

This invention relates to a method for processing and analysing samples and an apparatus for carrying out the method. In particular, the invention relates to a combination of sequential processing steps self-contained within a housing. The invention can be used in relation to any sample analysis where processing of the sample with one or more reagents is required. The invention has particular relevance for DNA analysis.

### BACKGROUND

In many fields of technology, samples of materials must be removed from a source and analysed, often following treatment with one or more chemical reagents or exposure to specific physical conditions. For example, there is a great need for the DNA analysis of biological samples in a wide variety of technology fields and industries.

The sample must first be procured from its source, the sample chemically and physically treated to prepare the DNA contained in the sample, the DNA then mixed with reagents to commence DNA-specific reactions (such as standard PCR methodology), and the products of such reactions analysed by physical or chemical methods. There are many known laboratory methods and devices available for achieving the individual steps, but none capable of performing the necessary and complex processes within a single device outside of the laboratory environment.

Further, it is often necessary to analyse a large number of samples, for example in food processing production lines. It is therefore desirable to use a sampling and analysis method that can be automated at least in part.

One problem associated with some sample processing and analysis techniques is the time required to obtain the results of analysis. For example, standard PCR analysis of a biological sample typically takes 2 to 6 hours. To meet the demands of systems and processes used in many industries, there is a need for a sampling and analysis method that is rapid.

The stringent requirements of health and safety, particularly in the human health and food industries, mean that cross-contamination between samples must not occur. It is therefore desirable to use a sampling and analysis apparatus that is not reusable and is disposable.

Further, it is desirable to analyse samples without high levels of technical skill and training required by operators. A fully self-contained apparatus having equipment and materials embedded within obviates the need for the high skill and experience levels needed by operators of known sampling and processing devices. A fully self-contained apparatus also has the advantage of being able to be stored for extended periods.

It is therefore an object of the invention to provide a method of sample analysis that overcomes at least in part any of the above mentioned problems or disadvantages, or to at least provide a useful alternative.

### STATEMENTS OF INVENTION

In one aspect of the invention there is provided a method for analysing a sample including:
a) introducing the sample into a conduit, where the conduit has at least one sample processing reagent which is encapsulated by a solid or semi-solid hydrophobic material;
b) applying heat so that the solid or semi-solid material liquefies;
c) causing the sample processing reagent to move along the conduit allowing the sample processing reagent to come into contact with the sample and react with the sample to form a product mixture and
d) detecting the presence, and optionally measuring the concentration, of a product in the product mixture.

The sample may be any sample of biological or non-biological chemical substances where processing with other reagents is needed prior to detection or measurement.

In a preferred embodiment of the invention the sample contains DNA. The invention is particularly suited to PCR analysis of the DNA. Typical examples of a sample include manufactured DNA samples and samples of blood, serum, saliva, urine, and milk, and an extract obtained from bone, faeces, fat, flesh, hair, skin, plant material, microbes, or microbial habitats.

The at least one sample processing reagent may be an encapsulated aqueous solution of one or more chemical or biochemical reagents required for DNA analysis including oligonucleotides, deoxynucleoside triphosphates and thermostable DNA polymerase.

Preferably the solid or semi-solid material is a wax or grease, for example paraffin, that is phase separated from any aqueous solution of sample, reagents, or products.

The detection step may use any suitable means for detecting or measuring the concentration of the product, but preferably uses an optoelectronic means.

Preferably the optoelectronic means is a light path between the sample and an optical detector which is connected to a microprocessor.

The at least one sample processing reagent is preferably caused to move along the conduit by the application of increased or reduced pressure to the conduit.

In a preferred embodiment of the invention a biological sample containing DNA is analysed according to the following steps:
1) the sample is introduced into one end of the conduit, where an aqueous solution of reagent encapsulated by a solid or semi-solid hydrophobic material and a lyophilised mixture of PCR reagents are contained within the conduit;
2) the conduit is heated so that the solid or semi-solid hydrophobic material liquefies releasing the aqueous solution of reagent, so that the aqueous solution of reagent contacts the lyophilised mixture of PCR reagents and rehydrates the PCR reagents so that an aqueous mixture of PCR reagents forms;
3) the aqueous mixture of PCR reagents is moved along the conduit in a forward direction, by the application of pressure to the conduit, so that the aqueous mixture of PCR reagents contacts the sample and mixes with the sample to form a sample mixture;
4) the sample mixture is then moved along the conduit in a reverse direction, by the application of reduced pressure to the conduit, so that the sample mixture lies in a denaturation zone where the mixture is heated to denature DNA contained in the sample;
5) the mixture containing denatured DNA and PCR reagents is then moved further along the conduit in the reverse direction, by the application of reduced pressure to the conduit, to an annealing zone and heat abstracted from the mixture to anneal oligonucleotide primers contained within the mixture to the denatured DNA;
6) the solution containing annealed DNA is moved along the conduit, by the application of pressure or reduced pressure to the conduit, to a detection zone and the DNA concentration determined optoelectronically;
7) the mixture containing annealed DNA and PCR reagents is then moved along the conduit in the forward direction, by the application of pressure to the conduit, to an extension zone and heat applied to cause extension of the annealed DNA by enzyme catalyzed DNA polymerization;
8) the mixture is then moved along the conduit in the reverse direction, by the application of reduced pressure to the conduit, so that the mixture lies in the denaturation zone where the mixture is heated to denature DNA contained in the sample;
9) the mixture is then moved further along the conduit in the reverse direction, by the application of reduced pressure to the conduit, to an annealing zone and heat abstracted to anneal oligonucleotide primers to the denatured DNA; and
10) the mixture is then moved along the conduit, by the application of pressure or reduced pressure to the conduit, to a detection zone and the DNA concentration determined optoelectronically.

Preferably the above steps 7 to 10 are repeated one or more times, typically 20-40 times.

In a second aspect of the invention there is provided an apparatus for analysing a sample including:
a) a sampling device having a shaft with a conduit located in the shaft where, in use, the sample is contacted with one or more sample processing reagents to give a product mixture;
b) a sample processing apparatus having a receptacle shaped to accommodate the shaft of the sampling device;
c) one or more heating elements located in the sample processing apparatus for heating one or more regions of the sampling device, when in use, to enable reaction of the sample with the one or more sample processing reagents to give the product mixture; and
d) a detection means to detect or measure one or more characteristics of a product in the product mixture.

In a preferred embodiment of the invention, the sampling device includes a free rolling ball held within a socket where part of the external surface of the ball is capable of contact with a surface to obtain a sample from that surface by rolling the ball across the surface.

Preferably the shaft of the sampling device is a tapered longitudinal stylus, having a sample inlet in open communication with the socket and a conduit extending the length of the stylus.

The invention further provides a sampling device adapted for use in the apparatus of the second aspect of this invention. The invention also provides a sample processing apparatus adapted for use in the apparatus of the second aspect of the invention.

### BRIEF DESCRIPTION OF THE FIGURES

FIG. 1 shows a sampling device of the invention in schematic form.

FIG. 2 shows a sample processing apparatus designed to accommodate the sampling device of FIG. 1.

FIG. 3 shows the sampling device of FIG. 1 located in the sample processing apparatus of FIG. 2 prior to heating.

FIG. 4 shows the sampling device of FIG. 1 located in the sample processing apparatus of FIG. 2 following heating

### DETAILED DESCRIPTION

This invention is based on the development of a method of sample analysis and an apparatus for carrying out the method where samples are processed with one or more reagents and the products detected in a manner where the sample and reagents and products can be manipulated by movement along a conduit to various zones that can be heated in a controlled way and where the reaction products are detectable.

The method includes the steps of:
a) introducing the sample into a conduit, where the conduit has at least one sample processing reagent which is encapsulated by a solid or semi-solid hydrophobic material;
b) applying heat so that the solid or semi-solid material liquefies;
c) causing the sample processing reagent to move along the conduit allowing the sample processing reagent to come into contact with the sample and react with the sample to form a product mixture; and
d) detecting the presence, and optionally measuring the concentration, of a product in the product mixture.

The method is useful for the analysis of a wide variety of sample types, especially where the sample must undergo one or more chemical transformations. The invention is most suited to the DNA analysis of samples, but is not limited to this use.

The sample may be introduced into the conduit by any suitable means. However, a preferred means is by using a free rolling ball held in a socket and rolling the ball across the surface from which the sample is to be obtained. A sampling device of this nature is the subject of the applicant's PCT patent application no. PCT/NZ04/0001 91.

The conduit has internal geometry that is preferably a single linear unbranched pathway and, although may be tapered, has no physical obstructions. However, the conduit may be non-linear or branched.

The sampling device may be constructed of any suitable material, but is preferably constructed from acrylic. The sampling device may alternatively be constructed from a hydrocarbon or perfluorocarbon plastic, a polymer resin, silicate (glass), silicon, doped silicon, or other semiconductor based materials, a metal, or a metal alloy. The ball is preferably constructed of the same material.

The conduit preferably has an internal surface that is coated, either prior to or during the course of sample processing, with an inert material such as a hydrocarbon, perfluorocarbon, or silicone wax, oil or grease.

The surface of the ball may be smooth, or may be textured or roughened, or coated with chemical modifiers, to minimise slippage of the ball on the surface when in use and/or to maximise or control sample adhesion.

Although a roller ball is the preferred means for obtaining a sample and delivering the sample to the conduit, other devices may be used, such as wheels, valves, vents, wicks, capillaries, syringes, pipettes, tweezers, probes, automated samplers, brushes, scalpels, needles, and fingers.

The sample may enter the conduit at atmospheric pressure, or under applied pressure, or by vacuum where the sample is sucked into the conduit.

The sample is preferably a laboratory prepared sample or biological material selected from the group including, but not limited to, blood, serum, saliva, urine, milk, and an extract obtained from bone, faeces, fat, flesh, hair, skin, plant material, microbes or microbial habitats. The sample may alternatively be a non-biological sample selected from the group including, but not limited to, water from waterways, industrial waste, and hazardous or non-hazardous chemicals including radioactive materials.

The one or more sample processing reagents may be any solid, fluid or gaseous reagent suitable for processing and/or analysis of the sample. In a preferred embodiment the one or more reagents are selected from the group, but not limited to hydrocarbon, perfluorocarbon or silicone greases waxes and oils; water based buffers and water solubilised organic and inorganic chemical reagents, trisma, magnesium chloride, potassium chloride, Bovine serum albumin (BSA), trehalose, ficoll, melizitose, sucrose, agarose; biochemical reagents required for enzymic DNA analysis including oligonucleotides, deoxynucleoside triphosphates and thermostable DNA polymerase.

Some or all of the one or more reagents may be hydrated or dehydrated by processes including, but not limited to, lyophilisation, evaporation, freeze drying or the like. For example, in the case of DNA analysis, the PCR reagents can be in the form of a lyophilised plug in the conduit of the sample device to maximize the stability of the reagents. During sample processing, the lyophilised plug is contacted with an aqueous solution causing the plug to rehydrate and be utilised in sample analysis.

In a preferred embodiment the conduit is open-ended allowing for pneumatic, hydrostatic, osmotic or electroosmotic control of samples, solutions of reagents and products, as well as for analytical interrogation.

The construction of the conduit may be by way of extrusion or pultrusion, such as in the formation of a tube, by machining (including drilling), by injection moulding, by injection compression moulding, by lithographic methods (including photon, electron or other particle based lithographies), by chemical etching, by contact or non-contact printing (including relief stamping), or by scratching, cutting or carving. As will be appreciated by those skilled in the art, manufacture of the apparatus of the invention is well suited to mass production, automation and miniaturisation.

The conduit passes through one or more temperature regulated zones and so facilitates temperature dependent processing of the sample and chemical reagents. The use of non-polar thermolabile chemical materials (such as waxes and greases) facilitates manipulation of the materials by changes in temperature to delay or initiate chemical activity within the lumen of the conduit. The appropriate arrangement of such materials enables long-term storage of prefabricated devices.

Surveillance and analysis of the conduit lumen can be performed by electronic (capacitive, inductive, resistive), optoelectronic (light scatter, refractive index, reflectance, absorbance, fluorescence, luminescence), ferro and para magnetic, resonance (nuclear magnetic, proton magnetic etc) methods. Such interrogation of the lumen can be performed transversely (through the lumen wall), axially (down the core of the lumen), or by a combination of both.

The processed sample can also be recovered from the lumen of the conduit for more detailed analysis. Such analysis could include chromatography, electrophoresis, spectroscopic techniques (mass, atomic, absorption etc), biological assay, macromolecular sequence analysis including nucleic acid and protein sequencing.

The invention has a number of important advantages. The fully self-contained chemistry requires no special technical knowledge by a user of the apparatus. No other materials, such as other reagents, are needed. The invention is well suited for use outside the laboratory environment. It is also easily adaptable to automation, including remote automation. The elimination of material transfer steps means that loss of sample is minimised.

The construction of the device is in two parts, the sampling device and the sample processing apparatus. This enables sophisticated processing and detection capability to reside in the sample processing apparatus while the sampling device, once used and contaminated with sample, can be disposable. The containment of sample processing reagents in the sampling device provides for an extended shelf life enabling ease of manufacture, distribution and storage. Further, the invention is amenable to miniaturization and mass production.

The invention also has the advantage of speed of sample processing. In the case of PCR analysis, the time taken from sampling to final result is typically less than 30 minutes

Other advantages will be apparent from reading this specification.

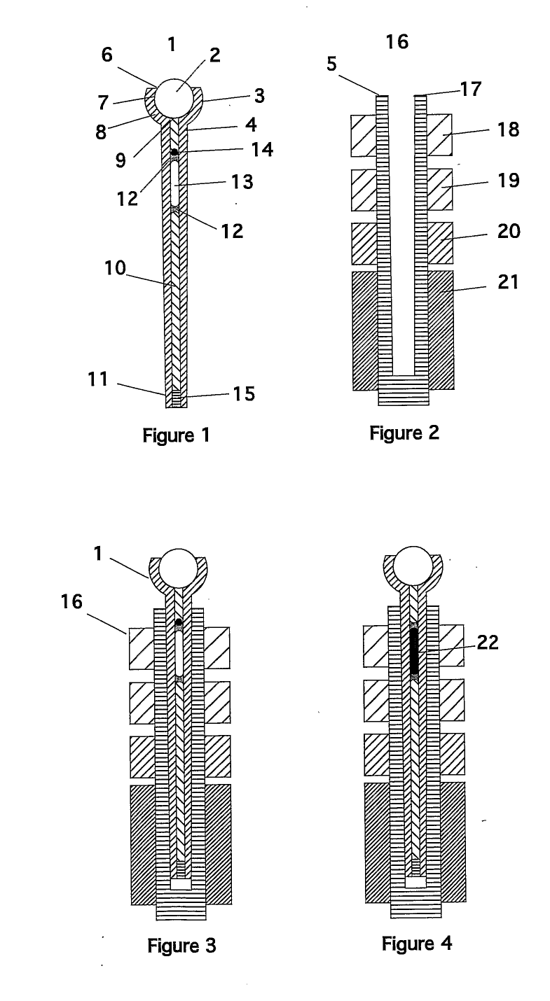

The invention will now be described by way of example with reference to FIGS. 1 to 4. It is to be appreciated that the device shown in FIGS. 1 to 4 is one example of the invention and that the invention is not limited to the example.

The cartridge device 1 comprises a sampling ball 2 housed within a socket 3. The ball 2 is free to rotate in any direction and has a surface that can range from substantially smooth to textured. The socket 3 is attached to or is integrally formed with the end of a shaft 4. The shaft 4 is tapered to facilitate easy entry to and exit from a receptacle 5 (as shown in FIGS. 2, 3 and 4) used for the analysis. As the processing of a sample requires heating, it will be appreciated that the device 1 will expand against the inner wall of the receptacle when heated. The taper of the shaft 4 also has the advantage of minimising the trapping of air when the device 1 is placed in the receptacle 5.

The shaft also serves as a handle. The shaft 4 may additionally be shaped or configured to suit the manner in which it is to be used, such as for holding by an operator during manual operation or for use by robotic manipulation.

The device 1 and the ball 2 may be constructed of any suitable material, typically acrylic or some other suitable plastics material.

The socket 3 comprises an opening 6 through which the ball is exposed and is accessible to a surface from which a sample may be obtained. The narrow gap between the ball 2 and the socket 3 represents the sample inlet 7. The diameter of the opening of the socket 3 is smaller than the diameter of the ball 2, thus preventing the ball 2 from falling out of the socket 3.

The inner wall 8 of the socket 3 opens behind the ball 2 to form a sample outlet 9, which is connected to the lumen 10 of the shaft 4. The lumen 10 extends the entire length of the shaft 4 and is a conduit through which the sample can be transported by applying reduced pressure (vacuum) or increased pressure to the lumen 10 at the distal end 11 of the shaft 4.

A series of plugs of paraffin 12 are located within the lumen 10. As will be appreciated by those skilled in the art, the plugs of paraffin 12 wet the inner surface of the lumen 10. The plugs of paraffin 12 form zones around an aliquot of aqueous material 13. It will be appreciated that paraffin can be used as a solid at room temperature, such as a wax or a grease, but as a fluid at elevated temperatures.

A lyophilised aliquot of reagents 14 is located within the lumen 10. For the purpose of PCR analysis, the lyophilised aliquot of reagents 14 contains a cocktail of biochemical reagents required including trisma, potassium chloride, magnesium chloride, Bovine serum albumin (BSA), trehalose, sucrose, melizitose, ficoll, oligonucleotides, deoxynucleoside triphosphates and thermostable DNA polymerase.

An aerosol filter 15 is also located within the lumen 10 at the distal end 11 of the shaft 4. The outer diameter of the aerosol filter 15 is such that it fits tightly within the lumen 10 and is held firmly. The aerosol filter 15 must be sufficiently porous to allow air to flow for the movement of samples and fluids within the lumen 10. The aerosol filter 15 is typically made from high molecular weight polypropylene.

Referring now to FIGS. 2 and 3, the processing apparatus 16 has a receptacle having a tapered internal wall 17 corresponding in shape to the tapered external wall of the shaft 4 of the device 1. The receptacle 5 is therefore shaped to receive the device 1 in a snug fit. The receptacle 5 may be constructed of any suitable material, but is typically made of Teflon, which is self-lubricating and therefore aids in the easy insertion and removal of the device 1.

The processing apparatus 16 also has three spatially separated heat zones 18, 19, and 20. The heat zones are in contact with the wall of the receptacle 5 and are able to transfer heat to the device 1 when inserted in the processing apparatus 16.

The heat zones 18, 19, and 20 may be constructed of any suitable material, but are preferably aluminium. The heat zones 18, 19, and 20 have embedded heating elements and temperature measuring elements to allow the setting, monitoring and control of the temperature of each of the heat zones 18, 19, and 20 independently from each other.

The heat zones 18, 19, and 20 are sufficiently spaced apart such that their temperature regulation is not coupled to each other. In other words, the temperature of one zone does not influence the temperature of another zone to any significant extent. The space may be any appropriate size, but is typically 3 mm.

For the processing of a sample containing DNA using PCR analysis, the heat zone 18 is set to 95° C. and is designated the denaturation zone. The heat zone 19 is set to 72° C. and is designated the extension zone. The heat zone 20 is set between the range 45-60° C. and is designated the annealing zone.

The heat zones 18, 19, and 20 will transfer heat to the receptacle 5 to create corresponding heat zones on the internal tapered wall 17 of the receptacle 5. Heat will then be transferred to the shaft 4 of the device 1 and to the lumen 10.

The heat zones 18, 19, and 20 are also fitted with a means to detect the presence of fluid plugs in the lumen 10. This may be by any suitable mechanism, but preferably by optoelectronic means.

The apparatus 16 also has a base unit 21, which houses a pneumatic pressure controller, a valve and a spectroscopic optoelectronic detector. The apparatus 16 also comprises, or is linked to, a microprocessor.

During operation the shaft 4 of the device 1 is held and the sampling ball 2 is rolled across the surface from which a sample containing the biological material of interest is to be analysed. Rotation of the ball 2 within the socket 3 causes sample to transfer from the ball 2 to the sample inlet 6. In addition, shear forces between the ball 2 and the socket 3 may help to facilitate disruption of cells in the sample.

The device 1 is then inserted into the receptacle 5 of the processing apparatus 16 as shown in FIG. 3. Upon insertion, a series of microprocessor controlled timed events facilitate the processing of the DNA by appropriate PCR chemistry, and the detection of the products of the DNA chemistry.

Firstly, the heat zones 18, 19, and 20 transfer heat to the lumen 10 of the shaft 4. The plugs of paraffin 12 then melt freeing the encapsulated aliquot of aqueous material 13 and enabling the lyophilised aliquot of reagents 14 to contact the freed aqueous material 13 and so rehydrate the lyophilised aliquot of reagents 14 to form a reagent mixture 22.
STEP 1 In a first step of processing of the sample the pressure controller delivers a metered pulse of pressure to the reagent mixture 22 vertically through the lumen 10. The position of the reagent mixture 22 is detected optoelectronically as it passes through the heat zones 18, 19, and 20. The reagent mixture 22 is shunted until it contacts the sample at the sample outlet 8 to form a mixture of sample and reagents 22.
STEP 2 Following contact of the sample with the reagent mixture 22, the pressure controller delivers a metered pulse of reduced pressure in the lumen 10 until the mixture of sample and reagents is withdrawn down the lumen 10 to the zone of denaturation block 18. The microprocessor ensures that the mixture of sample and reagents remains within the zone of the denaturation block 18 for a timed period. The mixture is heated at approximately 95° C. during this time. This may be any appropriate period, but is typically 1-2 minutes. This step serves to free much of the DNA from cellular material. PCR denaturation takes place.
STEP 3 Upon completion of the denaturation step, the pressure controller delivers a metered pulse of reduced pressure to the lumen 10 until the solution of the denatured DNA lies in the zone of annealing block 20. The microprocessor ensures that the time period in this zone is typically 8 seconds. This constitutes the annealing step of the PCR process. During this period, spectroscopic optoelectronic measurements determine the DNA concentration by means of fluorophore excitation, detection and quantification of the emitted fluorescence.
STEP 4 Following DNA concentration measurement, the pressure controller delivers a metered pulse of pressure to the lumen 10 until the solution lies in the zone of extension block 19. The microprocessor ensures that the solution remains within this zone for a timed period. This may be any appropriate period, but is typically 16 seconds. This constitutes the extension step of the PCR process.
STEP 5 The pressure controller then delivers a metered pulse of pressure to the lumen 9 until the solution lies in the zone of denaturation block 18. The time period for this denaturation step is typically 8 seconds.

The steps 3 to 5 constitute a complete cycle of PCR and an effective doubling of the target DNA sequences. Any number of cycles may be used, but typically 35 cycles are used. Real time analysis is achieved during each cycle at the annealing stage.

For the purpose of PCR analysis, the lyophilised aliquot of reagents 14 contains trisma, potassium chloride, Bovine serum albumin (BSA), trehalose, oligonucleotides, deoxynucleoside triphosphates and thermostable DNA polymerase.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### INDUSTRIAL APPLICABILITY

The method and apparatus of the invention are useful in a wide range of applications. The invention is particularly useful for the DNA testing of samples using PCR technology. However, the invention may be used for any sample processing where mixing with reagents is required before sample analysis. The invention has enhanced industrial application as it can be used out side the laboratory environment and does not need highly technical skilled operators.

1-27. (canceled)

28. A method for analysing a sample including:
a. introducing the sample into a conduit, where the conduit has at least one sample processing reagent which is encapsulated by a solid or semi-solid hydrophobic material;
b. applying heat so that the solid or semi-solid material liquefies;
c. causing the sample processing reagent to move along the conduit allowing the sample processing reagent to come into contact with the sample and react with the sample to form a product mixture; and
d. detecting the presence, and optionally measuring the concentration, of a product in the product mixture.

29. A method as claimed in claim 28 where the sample contains DNA.

30. A method as claimed in claim 29 where the sample is blood, serum, saliva, urine, or milk, or is an extract obtained from bone, faeces, fat, flesh, hair, skin, plant material, microbes, or microbial habitats.

31. A method as claimed in claim 28 where the at least one sample processing reagent is in aqueous solution or is a solid.

32. A method as claimed in claim 28 where the at least one sample processing reagent is a mixture of chemical and/or biochemical reagents.

33. A method as claimed in claim 28 where the at least one sample processing reagent is a mixture of chemical and/or biochemical reagents required for an enzyme assay.

34. A method as claimed in claim 28 where the at least one sample processing reagent is a mixture of chemical and/or biochemical reagents required for DNA analysis.

35. A method as claimed in claim 34 where the reagents required for DNA analysis include one or more of inorganic salts, tris buffer, bovine serum albumin (BSA), oligonucleotides, deoxynucleoside triphosphates, ficoll, sucrose, agarose, melizitose and thermostable DNA polymerase.

36. A method as claimed in claim 28 where the sample processing reagent is a lyophilised solid.

37. A method as claimed in claim 28 where the solid or semi-solid material is a wax or grease.

38. A method as claimed in claim 37 where the solid or semi-solid material is paraffin.

39. A method as claimed in claim 28 where detecting or measuring the concentration of the product in the product mixture is by an optoelectronic means.

40. A method as claimed in claim 39 where the optoelectronic means includes a light path between the product mixture and an optical detector.

41. A method as claimed in claim 40 where the optoelectronic means includes a light path projecting into an optical detector, where the optical detector is connected to a microprocessor.

42. A method as claimed in claim 28 where the sample and/or the sample processing reagent are caused to move along the conduit by the application pressure to the conduit.

43. A method of analysing a biological sample containing DNA according to the following steps:
1) the sample is introduced into one end of the conduit, where an aqueous solution of reagent encapsulated by a solid or semi-solid hydrophobic material and a lyophilised mixture of PCR reagents are contained within the conduit;
2) the conduit is heated so that the solid or semi-solid hydrophobic material liquefies releasing the aqueous solution of reagent, so that the aqueous solution of reagent contacts the lyophilised mixture of PCR reagents and rehydrates the PCR reagents so that an aqueous mixture of PCR reagents forms;
3) the aqueous mixture of PCR reagents is moved along the conduit in a forward direction, by the application of pressure to the conduit, so that the aqueous mixture of PCR reagents contacts the sample and mixes with the sample to form a sample mixture;
4) the sample mixture is then moved along the conduit in a reverse direction, by the application of reduced pressure to the conduit, so that the sample mixture lies in a denaturation zone where the mixture is heated to denature DNA contained in the sample;
5) the mixture containing denatured DNA and PCR reagents is then moved further along the conduit in the reverse direction, by the application of reduced pressure to the conduit, to an annealing zone and heat abstracted from the mixture to anneal oligonucleotide primers contained within the mixture to the denatured DNA;
6) the solution containing annealed DNA is moved along the conduit by the application of pressure or reduced pressure to the conduit, to a detection zone and the DNA concentration determined optoelectronically;
7) the sample mixture containing annealed DNA and PCR reagents is then moved along the conduit in the forward direction, by the application of pressure to the conduit, to an extension zone and heat applied to cause extension of the annealed DNA by enzyme catalyzed DNA polymerization;
8) the mixture is then moved along the conduit in the reverse direction, by the application of reduced pressure to the conduit, so that the mixture lies in the denaturation zone where the mixture is heated to denature DNA contained in the sample;
9) the mixture is then moved further along the conduit in the reverse direction, by the application of reduced pressure to the conduit, to an annealing zone and heat abstracted to anneal oligonucleotide primers to the denatured DNA; and
10) the mixture is then moved along the conduit, by the application of pressure or reduced pressure to the conduit, and the DNA concentration determined optoelectronically.

44. A method as claimed in claim 43 where steps 7 to 10 are repeated one or more times.

45. An apparatus for analysing a sample including:
a) a sampling device having a shaft with a conduit located in the shaft where, in use, the sample is contacted with one or more sample processing reagents to give a product mixture;
b) a sample processing apparatus having a receptacle shaped to accommodate the shaft of the sampling device;
c) one or more heating elements located in the sample processing apparatus for heating one or more regions of the sampling device, when in use, to enable reaction of the sample with the one or more sample processing reagents to give the product mixture; and
d) a detection means to detect or measure one or more characteristics of a product in the product mixture.

46. An apparatus as claimed in claim 45 where the sampling device includes a free rolling ball held within a socket where part of the external surface of the ball is capable of contact with a surface to obtain a sample from that surface by rolling the ball across the surface.

47. An apparatus as claimed in claim 46 where the shaft of the sampling device is a tapered longitudinal stylus, having a sample inlet in open communication with the socket and a conduit extending the length of the stylus.

48. An apparatus as claimed in claim 47 where shaft of the sampling device provides an optical path for analysing the product.

49. An apparatus as claimed in claim 45 where the detection means includes a transparent window in the wall of the sampling device.

50. A sampling device adapted for use in the apparatus as claimed in claim 45.

51. A sample processing apparatus adapted for use in the apparatus of claim 45.
